(19) United States
(12) Patent Application Publication
(10) Pub. No.: US 2017/0074860 A1
(43) Pub. Date: Mar. 16, 2017

(54) THREE-DIMENSIONAL CANCER CULTURE MODEL

(71) Applicant: THE TRUSTEES OF COLUMBIA UNIVERSITY IN THE CITY OF NEW YORK, New York, NY (US)

(72) Inventor: Daniel S. Oh, Northvale, NJ (US)

(21) Appl. No.: 15/276,155

(22) Filed: Sep. 26, 2016

Related U.S. Application Data
(63) Continuation of application No. PCT/US15/22941, filed on Mar. 27, 2015.
(60) Provisional application No. 61/971,221, filed on Mar. 27, 2014.

(51) Int. Cl. G01N 33/50

(52) U.S. Cl. CPC G01N 33/5011

(57) ABSTRACT

A three-dimensional cancer culture model is provided suitable for chemotherapeutic testing and metastasis mechanism study.

### CROSS-REFERENCE TO RELATED APPLICATIONS

This application is a continuation of International Application No. PCT/US15/22941, filed Mar. 27, 2015, which claims the benefit of U.S. Provisional Application No. 61/971,221, filed Mar. 27, 2014, both of which are herein incorporated by reference in their entirety.

### BACKGROUND OF THE DISCLOSED SUBJECT MATTER

Field

The present disclosure generally relates to a three-dimensional cancer culture model, and more particularly to a bioreactor including a three-dimensional scaffold suitable for the culture of cancer cells for chemotherapeutic agents testing and metastasis mechanism study.

Background

Two-dimensional (2D) cancer model systems are available to investigate disease mechanisms and to screen therapies. While these models have contributed information about cancer biology, these simplistic models fail to adequately model the in vivo environment. Approximately 90% of promising preclinical drugs, in all therapeutic classes, fail to result in efficacious human treatments, thereby wasting vast amounts of time and money and, ultimately, delaying the discovery of successful interventions. Two-dimensional tissue culture models lack realistic complexity, while animal models are expensive, time consuming, and too frequently fail to reflect human tumor biology. These 2D culture systems do not reflect the true three-dimensional (3D) microenvironment present in human tissues and/or tumors, whereby cell-cell and cell-extracellular matrix (ECM) interactions occur.

Thus, there remains a need in the art for a 3D microenvironment suitable for the study of cancer cell proliferation, motility and differentiation.

### SUMMARY

The present disclosure relates to systems, methods, and apparatus for cancer culture. The subject matter of the present disclosure is suitable for chemotherapeutic agents testing and metastasis mechanism study. In addition, it is suitable for testing of other therapies that require a 3D microenvironment simulating the in-vivo cancer growth.

In one aspect, a bioreactor is provided. The bioreactor includes a culture medium and a scaffold. The scaffold is immersed in the culture medium. The scaffold has a plurality of macro-pores and a plurality of nano-pores.

In some embodiments, substantially all of the macro-pores have a diameter between about 150 μm and about 650 μm. In some such embodiments, substantially all of the macro-pores have a diameter between about 200 μm and about 400 μm. In some embodiments, substantially all of the nano-pores have a diameter between about 100 nm and 400 nm. In some embodiments, the plurality of macro-pores are interconnected.

In some embodiments, the scaffold further comprises a plurality of micro-channels. In some embodiments, substantially all of the micro-channels have a diameter between about 25 μm and 70 μm. In some embodiments, the micro-channels are interconnected.

In some embodiments, the scaffold has thereon a plurality of cells. In some embodiments, the calls are selected from the group consisting of: osteoblasts; osteoblast precursors; fibroblasts; muscle cells; bone marrow cells; and mesenchymal stem cells. In some embodiments, the cells are selected from the group consisting of: osteosarcoma cells, chondrosarcoma cells, Ewing's sarcoma cells, fibrosarcoma cells; carcinoma cells; and breast cancer cells. In some embodiments, the cells are distributed substantially evening throughout the scaffold. In some embodiments, the scaffold has an interior region and the interior region has hypoxic cells.

In some embodiments, the bioreactor includes a perfusion pump operable to circulate the culture medium. In some embodiments, the culture medium comprises a pharmaceutical. In some embodiments, the pharmaceutical is a chemotherapeutic agent.

In some embodiments, the scaffold is substantially cylindrical. In other embodiments, the scaffold is substantially spherical. In some embodiments, the scaffold has a diameter of about 8 mm. In other embodiments, the scaffold has a diameter of about 100 In some embodiments, the scaffold has a height of about 8 mm. In yet other embodiments, the scaffold is substantially cuboidal.

It is to be understood that both the foregoing general description and the following detailed description are exemplary and are intended to provide further explanation of the disclosed subject matter claimed. The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part. Similar reference numerals (differentiated by the leading numeral) may be provided among the various views and Figures presented herein to denote functionally corresponding, but not necessarily identical structures.

FIG. 1 depicts a bioreactor system according to embodiments of the present disclosure.

FIG. 2A is a side view of an exemplary cylindrical cancer culture scaffold according to embodiments of the present disclosure.

FIG. 2B is a top view of an exemplary spherical cancer culture scaffold according to embodiments of the present disclosure.

FIGS. 3A-D depict the macro-, micro-, and nano-structure of an exemplary cancer culture scaffold according to embodiments of the present disclosure.

### DETAILED DESCRIPTION OF EXEMPLARY EMBODIMENTS

Reference will now be made in detail to exemplary embodiments of the disclosed subject matter, an example of which is illustrated in the accompanying drawings. Methods and corresponding steps of the disclosed subject matter will be described in conjunction with the detailed description of the system.

A 3D microenvironment according to embodiments of the present disclosure is suitable for the study of cancer cell proliferation, motility and differentiation. According to various embodiments of the present disclosure, an engineered 3D culture platform is provided that comprises a scaffold having a three-leveled micro-architecture. The scaffold includes interconnected macro-pores that mimic trabecular bone. In some embodiments, the macro-pores are about 300-400 μm in diameter. Although various exemplary embodiments are described with reference to the trabecula, the platform and microenvironment of the present disclosure is suitable for simulation of various in vivo environments. The scaffold also includes micro-channels within the trabecular structure. In some embodiments, the micro-channels are about 25-70 μm in diameter. The scaffold also contains nano-pores on its surface. In some embodiments, the nano-pores are about 100-400 nm in diameter.

Various scaffolds may be used as part of the platform and bioreactor of the present disclosure. In some embodiments, the scaffold described in commonly invented U.S. Patent Pub. No. 2011/0313538 is used. Said application, entitled Bi-Layered Bone-Like Scaffolds, is hereby incorporated by reference in its entirety.

In various embodiments of the present disclosure, the scaffold is placed in a perfusion bioreactor. Such combined systems provide a highly porous structure, containing multiple microenvironments. These microenvironments are suitable for ensuring the vitality and 3D growth of cancer cells in a 3D culture.

### Bioreactor System

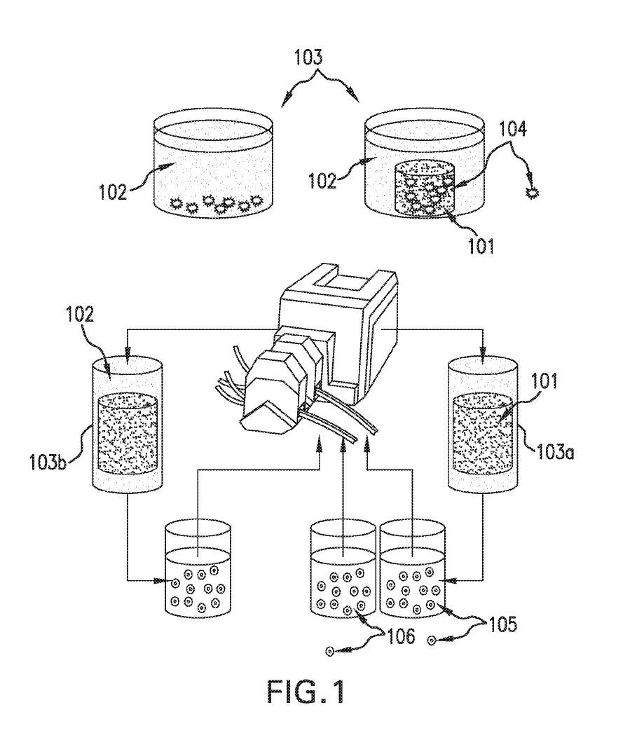

Referring now to FIG. 1, a bioreactor system suitable for the testing of therapeutic agents is depicted according to embodiments of the present disclosure. Scaffold 101 is suspended in culture medium 102 within vessel 103. Scaffold 101 is seeded with cells 104 under static culture conditions. In an exemplary embodiment, cells 104 are osteoblast precursors. However, the present subject matter is suitable for culture of various cells including precursors such as mesenchymal stem cells as well as chondrocytes, osteoblasts, and cancer cells including osteosarcoma, chondrosarcoma, Ewing's sarcoma, fibrosarcoma and carcinomas.

In some embodiments, vessel 103 is included in a bioreactor. Various bioreactor configurations are known in the art, and may be used in combination with the present subject matter. In various exemplary embodiments, the bioreactor includes one or more of: an agitation system, a feeding pump, an effluent pump, an air pump, an aerator, a sensor probe, a system monitor, and a thermal jacket. In alternative embodiments, a disposable bag or tube is used instead of culture vessel 103, providing a single use bioreactor.

In some embodiments, scaffold 101 is seeded with various cells, including but not limited to osteoblasts, osteoblast precursor cell lines, fibroblasts, muscle cells, bone marrow, and mesenchymal stem cells. In some embodiments, the cell line is MC3T3. In some embodiments, scaffold 101 is seeded with a combination of cells, including both healthy cells and cancerous cells.

In some embodiments, pre-cultured scaffold 101 is suspended in culture medium 102 within vessel 103a and 103b. Cells 105 are circulated through pre-cultured scaffold 101 using the bioreactor system. In an exemplary embodiment, cells 105 are osteosarcoma cells. However, the present subject matter is suitable for culture of various cancers including osteosarcoma, chondrosarcoma, Ewing's sarcoma, fibrosarcoma, carcinomas, and breast cancer cells.

In some embodiments, pharmaceutical testing is performed using treated vessel 103a and untreated vessel 103b. A pharmaceutical is introduced into vessel 103a, while vessel 103b is used as a control. As an example, Doxorubicin 106 can be perfused into the bioreactor to test drug resistance. As compared to a 2D surface, the platform of the present disclosure exhibits greater 3D cell mass and chemotherapy drug resistance.

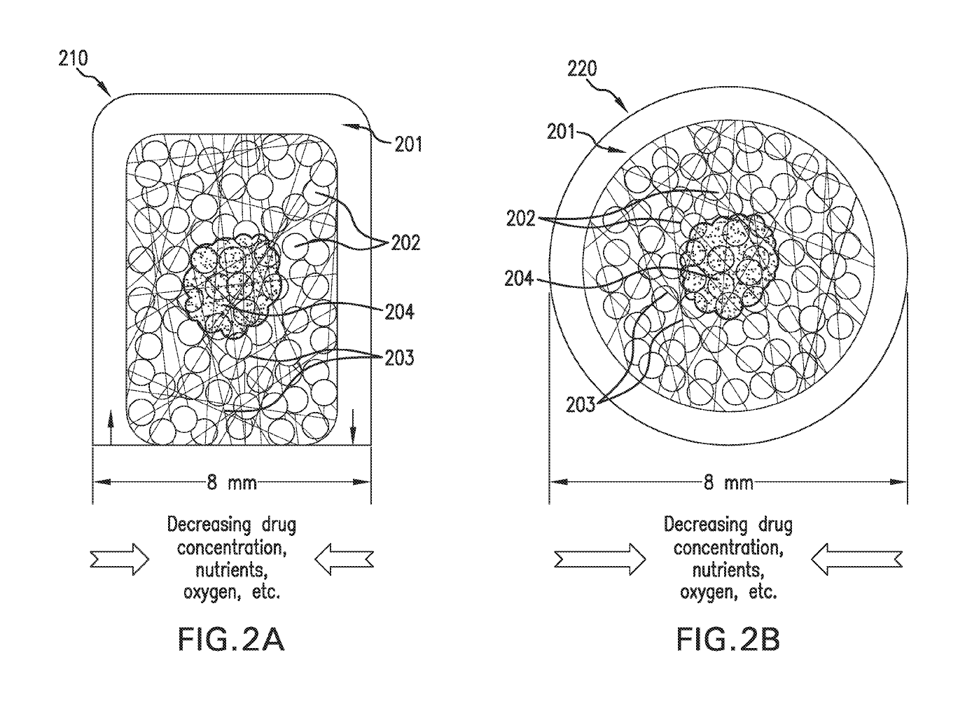

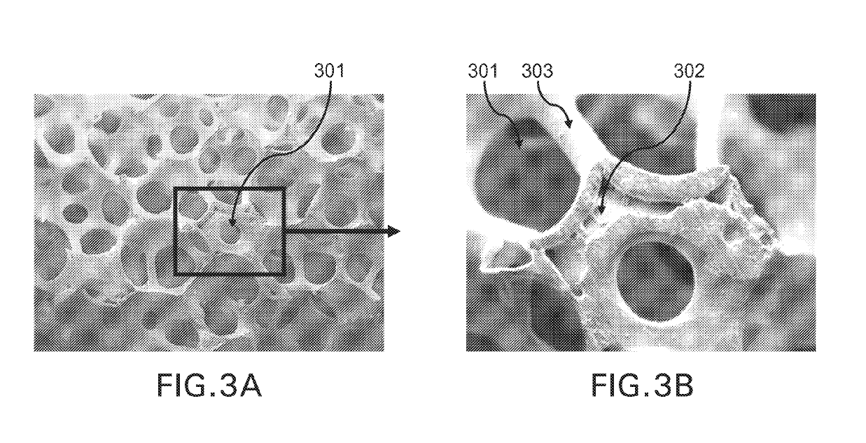

Referring now to FIG. 2, exemplary tumor cell niches according to various embodiments of the present disclosure are depicted. FIG. 2A is a side view of an exemplary cylindrical cancer culture scaffold 210. FIG. 2B is a top view of an exemplary spherical cancer culture scaffold 220. In some embodiments, scaffolds 210, 220 are about 8 mm in diameter. Other embodiments may have other diameters, such as 100 Circulation region 201 may be perfused with blood or culture medium. In in vivo environments, circulation region 201 may be a blood vessel. Tumor cells 202 grow on extracellular matrix 203.

Hypoxia is a crucial barrier to the delivery of chemotherapeutic agents. According to embodiments of the present disclosure, tumor hypoxia is induced by the characteristics of the scaffold, resulting in a necrotic region 204 near the center of the scaffold. The multi-leveled organization of the 3D construct within a perfusion bioreactor system demonstrates deteriorated microenvironments. The reduction of drug concentration, nutrients and oxygen creates a hypoxic environment near the center, in which oxygen content is low, but sufficient to keep cells alive in the middle zone of scaffold. Low nutrition, and acidosis incarnate in vivo tumor hypoxia. By virtue of this favorable microenvironment, the cancer cells are aggressively migrated and invaded into the engineered bone-like matrix.

The subject matter of the present disclosure is suitable for investigating the mechanism of bone metastasis of cancers such as breast cancer. In an exemplary embodiment, primary breast cancer cells are diluted in a media reservoir and the reservoir is connected to a pre-organized bone-like matrix scaffold column. With this configuration, the breast cancer cells are circulating through the engineered bone-like matrix to mimic physiological complications. The growth environment is suitable for study of cell-cell interaction and signaling pathways is favorable. This configuration allows generation of more predictive pre-clinical models to enhance cancer treatment efficacy.

### Scaffold

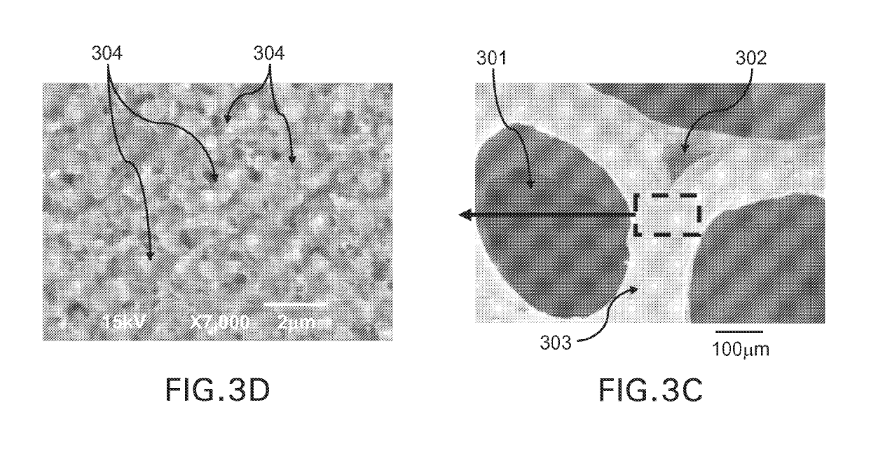

Referring now to FIG. 3, an exemplary scaffold is depicted. FIG. 3A depicts a plurality of macro-pores 301. FIG. 3B provides a zoomed in a view of a region of FIG. 3A, showing macro-pore 301, and micro-channels 302. FIG. 3C provides a further zoomed in view of a region of FIG. 3B, showing macro-pore 301, micro-channel 302, and trabecular beam 303. FIG. 3D provides a further zoomed in view of a region of FIG. 3C, showing nano-pores 304.

Scaffolds suitable for use according to the present disclosure generally exhibit biocompatibility, have closely matched mechanical properties when compared to native bone, and possess a mechanism to allow diffusion and/or transport of ions, nutrients, and wastes. The architecture of the scaffolds (pore size, porosity, interconnectivity and permeability suitable for ion and transport/diffusion of nutrients and wastes) allows sustained cell proliferation and differentiation within the scaffolds.

Scaffolds according to the present disclosure can have various shapes. Non-limiting examples of such shapes include cylinder, block, morsel, wedge, and sheet. The scaffold may be fabricated to simulate the hip, the femoral or humeral head or shaft, the femoral head surface or total joint, the vertebral column, the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones, or metatarsal bones.

In some embodiments, the scaffold of the present disclosure is a single-density or multi-density porous structure that promotes cellular and/or nutrient infiltration. Macro-pores and micro-channels support the in-growth of cells.

In some embodiments, the scaffold has an outer cortical shell and an inner trabecular core. The structure of such scaffolds resembles the structure of a long bone. Such a structure allows the outer cortical shell to be load bearing, as in native bone.

In other embodiments, the scaffold include a body having a long axis, wherein the scaffold has an open pore structure of micro-pores that are interconnected and secondary micro-channels that are generally perpendicular to the long axis of the body.

A micro-pore according to various embodiments is a small opening or passageway, having an average diameter of about 1 μm to about 3 mm. For example, a micro-pore may have an average diameter of about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 230, 240, 250, 260, 270, 280, 290, 300, 325, 350, 375, 400, 425, 450, 475, 500, 525, 550, 575, 600, 625, 650, 675,700, 725, 750, 775, 800, 825, 850, 875, 900, 925, 950, 975,1000, 1100,1200, 1300, 1400, 1500, 1600, 1700, 1800, 1900, 2000, 2100, 2200, 2300, 2400, 2500, 2600, 2700, 2900, or 3000 μm or more, or any range derivable therein. Micro-pores may or may not be connected to other micro-pores.

In those embodiments where the scaffold possesses interconnected micro-channels and/or micro-pores, all or only a portion of the scaffold may possess the micro-channels and/or micro-pores. In some embodiments, the micro-channels connect to micro-pores, while in some embodiments they do not.

In some embodiments, the micro-pores are of uniform shape, while in some embodiments they are distinctly shaped. In some embodiments, the micro-pores are of uniform size, while in other embodiments they are of a variety of sizes. They may be generally round, oval, cylindrical, or irregularly shaped. A micro-pore may be interconnected with one or more other micro-pores or one or more micro-channels. In some embodiments, the scaffold includes latent pores that become actual pores after the scaffold is placed in a perfusion bioreactor as described herein.

A micro-channel according to various embodiments of the present disclosure is a passageway that has an average diameter of about 1 μm to about 3 mm, wherein the length of the passageway is at least twice as long as the average diameter of the passageway. For example, a micro-channel may have an average diameter of about 1, 5, 10, 15, 20,25,30, 35, 40, 45, 50, 55, 60, 65, 70, 75, 80, 85, 90, 95, 100, 110, 120, 130, 140, 150, 160, 170, 180, 190, 200, 220, 230, 240,250, 260, 270, 280, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300,1400, 1500, 1600, 1700, 1800, 1900, 2000, 2100, 2200, 2300, 2400, 2500, 2600, 2700, 2900, or 3000 μm or more, or any range derivable therein. The micro-channel may have any average length. The length of micro-channels may vary with the size and shape of the scaffold.

A micro-channel may be interconnected with one or more other micro-channels or with one or more micro-pores. In embodiments of the present disclosure that possess an outer cortical shell and an inner trabecular core, the outer cortical shell and/or inner trabecular core may possess one or more micro-channels or micro-pores. Micro-channels and/or micro-pores of the outer cortical layer may be connected to micro-channels and/or micro-pores of an inner trabecular core. An interconnected structure of micro-pores and/or micro-channels allows for the transport of nutrients, ions, and/or cells. In some embodiments, only an outer cortical shell possesses micro-pores and/or micro-channels. In other embodiments, only an inner trabecular core possesses micro-pores and/or micro-channels. In some embodiments, both the trabecular core and the outer cortical shell possess micro-pores and/or micro-channels.

In some embodiments, the scaffold is cylindrical in shape and includes an outer cortical shell and inner trabecular layer to resemble the native structure of a portion of a long bone. Some embodiments of such scaffolds possess interconnected secondary micro-channels in a radial orientation within struts of the scaffolds in order to provide nutrients and ions to the interior of the structure. The strut is the main frame of the scaffold structure. The strut may comprise micro-channels.

In some embodiments, the scaffold includes (a) a core component having interconnected micro-pores; and (b) a cortical layer in contact with at least a portion of a surface of the core component, wherein the cortical layer comprises micro-pores and/or micro-channels. In some embodiments, the micro-pores of the core component are interconnected, which allows for the transport of nutrients and ions. In further embodiments, the micro-pores of the cortical layer are interconnected. In yet further embodiments, the micro-pores of the core component are interconnected with the micro-pores of the cortical layer.

In some embodiments, the micro-pores of the cortical layer have an average diameter that is less than the average diameter of the micro-pores of the core component. For example, in some embodiments, the core component comprises two populations of micro-pores, the first population of micro-pores having an average diameter of about 50 μm to about 1000 μm, and the second population of micro-pore having an average diameter of about 10 μm to about 300 μm. In some embodiments, the first type of micro-pore has an average diameter of about 150 μm to about 750 μm, and the second type of micro-pore has an average diameter of about 50 μm to about 120 μm. In some embodiments, the average diameter of the micro-pores of the cortical layer is about 1 μm to about 300 μm. In some embodiments, the average diameter of the micro-pores of the cortical layer is about 10 μm to about 150 μm.

The scaffold composite may be of any density. For example, the density may be about 5, 4.5, 4.0, 3.5, 3.0, 2.5, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 g/cm³, or any range of densities derivable therein. In some embodiments, the density is between about 0.05 g/cm³and about 1.60 g/cm³. In some embodiments, the porous composite has a density of between about 0.07 g/cm³and 1.1 g/cm³. The density may be less than about 1 g/cm³, less than about g/cm³, less than about 0. g/cm³, less than about 0.7 g/cm³, less than about 0.6 g/cm³, less than about 0.50 g/cm³, less than about 0.4 g/cm³, less than about 0.3 g/cm³, less than about 0.2 g/cm³, or less than about 0.1 g/cm³.

In embodiments of the present scaffolds that include a porous component, the porous component is of any porosity. For example, the porosity may be at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, or any range of porosities derivable herein. The core component and cortical layer of the scaffold can be of any porosity, including any of the porosities set forth above. In particular embodiments, the core component average porosity is 65% to 90% and cortical layer of the scaffold average porosity is 30% to 60%.

The scaffold can be of any shape and configuration. For example, in particular embodiments, the scaffold is cylindrical, thus resembling a long bone. In other embodiments, the scaffold is round, square, or of an irregular shape or comprised of granules of a size smaller than the bony defect they will be used to treat.

In some embodiments, the cortical layer comprises micro-channels. For example, in scaffolds with a cylindrical shape with a long axis, the secondary micro-channels have an axis that is generally perpendicular to the long axis of the scaffold. There can be any number of micro-channels in the cortical structure. In some embodiments, the secondary micro-channels have an average diameter that is greater than the average diameter of the micro-pores in the cortical layer. In particular embodiments, the secondary micro-channels have an average diameter of about 10 μm to about 500 μm. In some embodiments, the secondary micro-channels have an average diameter of about 50 μm to about 120 μm.

The core component may include a single population of micro-pores of uniform size and shape, or may include more than one population of micro-pores. In some embodiments, the first population of micro-pores has an average diameter of about 150 μm to about 750 μm, and the second population of micro-pores has an average diameter of about 50 μm to about 120 μm, wherein the average diameter of the micro-pores of the cortical layer is about 10 μm to about 150 μm.

The scaffold may be composed of a single type of material, or more than one material. In scaffolds that include more than one component, such as a scaffold that includes an inner trabecular core and outer cortical layer, the components of the scaffold may be composed of similar materials or different materials. The scaffold may be composed of more than one material, or a composite of materials.

In some embodiments, the scaffold includes calcium and phosphorus. For example, the calcium phosphate may be tricalcium phosphate, hydroxyapatite, amorphous calcium phosphate, monocalcium phosphate, dicalcium phosphate, octacalcium phosphate, tetracalcium phosphate, fluorapatite, carbonated apatite, an analog thereof, or a mixture thereof. The scaffold may be composed of a composition that includes calcium and phosphate (a calcium phosphate). A “calcium phosphate” as used herein is generally defined as any molecule that includes one or more calcium atoms, one or more phosphorus atoms, and one or more oxygen atoms. [0051] The scaffold may include one or more additional components. Examples include therapeutic agents, such as small molecules, polypeptides, proteins, DNA, RNA, antibodies, antibody fragments, metal ions (such as zinc or silver), and so forth. In some embodiments the therapeutic agent is an angiogenic factor or an osteogenic growth factor.

In some embodiments, the scaffold may further include particles. The particles in the composite may have a variety of shapes including spheroidal, plate, fiber, cuboidal, sheet, rod, ellipsoidal, string, elongated, polyhedral, and mixtures thereof. The particles in the composite may be of any size. For example, they may have an average size of about 10, 20, 30, 40, 50, 60, 70, 80, 90, 100, 150, 200, 250, 300, 350, 400, 450, 500, 550, 600, 650, 700, 750, 800, 850, 900, or 1000 microns in diameter, or any range of diameter derivable therein. In particular embodiments, the average particle size is about 20 to about 800 microns in diameter. Particles of varying sizes may be present within the same scaffold.

In some embodiments, channels are created in the sides of the scaffold into which beads that include one or more therapeutic agents can be placed. The beads can be coated with one or more therapeutic agents, or the therapeutic agents can be incorporated into the structure of the bead. The bead may or may not be resorbable. In some embodiments, the beads are composed of a polymer, such as any of those polymers set forth herein, or are ceramic. The channels, which may be larger than microchannels as described herein, can be created using any method known to those of ordinary skill in the art. In some embodiments, the channels are created by drilling into the side of the scaffold.

In some embodiments, the scaffold that is formed includes an inner core and an outer cortical layer. In some embodiments, the core component has an open pore structure of micro-pores that are interconnected. The cortical layer is in contact with at least a portion of the core component. In some embodiments, the cortical layer includes micro-pores.

In some embodiments, a biologically active substance is integrated into the scaffold and/or into a coating applied to the scaffold, or coating the inner aspect of the micro-pores of the scaffold. Thus, a controlled delivery of the biologically active substance is enabled. The amount of the biologically active substance may easily be defined by controlling the coating process, for example. By integrating biologically active substance into a submerged coating layer or region, or into the composition, a controlled retarded release of the biologically active substance may be accomplished. The biologically active substance can also be encapsulated in biodegradable microspheres or polymeric scaffolds and incorporated into channels of the scaffold using any method known to those of ordinary skill in the art, or incorporated into a particle.

Scaffolds according to the present disclosure may be composed of a variety of components. The components can be obtained from natural sources, commercial sources, or can be chemically synthesized. In some embodiments, the scaffold includes a calcium phosphate. Regarding natural sources, calcium phosphates are found in bone, teeth and shells of a large variety of animals. It exists in a variety of forms known in the art, and non-limiting examples include hydroxyapatite (Hydroxyapatite, Ca.sub.10(PO.sub.4).sub.6(OH).sub.2, Ca/P=1.67), tricalcium phosphate (TCP, Ca.sub.3(PO.sub.4).sub.2, Ca/P=1.5) and brushite (CaHPO.sub.4.2H.sub.20, Ca/P=1. Hydroxyapatite has characteristics similar to mineralized matrix of natural bone, and is biocompatible. Non-limiting examples of calcium compounds include calcium nitrate tetrahydrate, calcium nitrate, and calcium chloride. Non-limiting examples of phosphorus compounds include triethylphosphate, sodium phosphate, and ammonium phosphate dibasic. One of ordinary skill in the art would be familiar with the wide variety of calcium phosphates known in the art, and sources of such compounds.

The scaffolds of the present disclosure may include any component known to those of ordinary skill in the art to be suitable for inclusion in a biomedical scaffold. Other non-limiting examples of such components include polymethylmethacrylate (PMMA), calcium sulfate compounds, calcium aluminate compounds, aluminum silicate compounds, bioceramic materials, or polymers. Examples of the bioceramic material include calcium phosphate-based oxide, such as apatite, BIOGLASS™, glass oxide, titania, zirconia, and alumina. Other suitable materials include alginate, chitosan, coral, agarose, fibrin, collagen, bone, silicone, cartilage, aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, a-tricalcium phosphate, a dicalcium phosphate, β-tricalcium phosphate, tetracalcium phosphate, octacalcium phosphate (OCP), fluoroapatite, chloroapatite, magnesium-substituted tricalcium phosphate, carbonate hydroxyapatite, and combinations and derivative thereof. Examples of silicon compounds include tetraethylorthosilicate, 3-mercaptopropyltrimethoxysilane, and 5,6-epoxyhexyltriethoxysilane.

The scaffolds of the present disclosure may optionally include any number of additional additives. In some embodiments, additives are added to a portion of the scaffold. For example, a scaffold may include additives in the cortical shell but not in the inner trabecular core, or vice versa. In some embodiments, there are additives in both the cortical shell and trabecular core. Non-limiting examples of additives include radiocontrast media to aid in visualizing the scaffold with imaging equipment. Examples of radiocontrast materials include barium sulfate, tungsten, tantalum, or titanium. Additives that include osteoinductive materials may be added to promote bone growth into the hardened bone augmentation material. Suitable osteoinductive materials may include proteins from transforming growth factor (TGF) beta superfamily, or bone-morphogenic proteins, such as BMP2 or BMP7.

Useful non-erodible polymers include without limitation, polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, TEFLON™, nylon, stainless steel, cobalt chrome, titanium and titanium alloys, and bioinert ceramic particles (e.g., alumina and zirconia particles), polyethylene, polyvinylacetate, polymethylmethacrylate, silicone, polyethylene oxide, polyethylene glycol, polyurethanes, and natural biopolymers (e.g., cellulose particles, chitin, keratin, silk, and collagen particles), and fluorinated polymers and copolymers (e.g., polyvinylidene fluoride).

In some embodiments, the scaffold is coated with compounds to facilitate attachment of cells to the scaffold. Examples of such compounds include basement membrane components, agar, agarose, gelatin, gum arabic, collagens types I, II, III, IV, and V, fibronectin, laminin, glycosaminoglycans, polyvinyl alcohol, and mixtures thereof

In some embodiments, mammalian cells are incorporated into the scaffolds. Examples of such cells include, but are not limited to, bone marrow cells, smooth muscle cells, stromal cells, stem cells, mesenchymal stem cells, synovial derived stem cells, embryonic stem cells, umbilical cord blood cells, umbilical Wharton's jelly cells, blood vessel cells, chondrocytes, osteoblasts, osteoclasts, precursor cells derived from adipose tissue, bone marrow derived progenitor cells, kidney cells, intestinal cells, islets, beta cells, pancreatic ductal progenitor cells, Sertoli cells, peripheral blood progenitor cells, fibroblasts, glomus cells, keratinocytes, nucleus pulposus cells, annulus fibrosus cells, fibrochondrocytes, stem cells isolated from adult tissue, oval cells, neuronal stem cells, glial cells, macrophages and genetically transformed cells or combination of the above cells. The cells can be seeded on the scaffolds for a short period of time prior to use in a bioreactor (such as one hour, six hours, 24 hours), or cultured for longer periods of time (such as 2 days, 3 days, 5 days, 1 week, 2 weeks) to promote cell proliferation and attachment within the scaffold prior to testing.

Various methods are known in the art for fabrication of scaffolds suitable for use according to embodiments of the present disclosure. These include, without limitation, leaching processes, gas foaming processing, supercritical carbon dioxide processing, sintering, phase transformation, freeze-drying, cross-linking, molding, porogen melting, polymerization, melt-blowing, and salt fusion. In some embodiments, microchannels and/or larger channels are drilled into the scaffold following molding.

The scaffolds set forth herein can be formed into a desired shape using any method known to those of ordinary skill in the art. For example, the scaffold may be molded into a desired shape or fractured into granules. The granules retain the essential micropores and/or microchannels. The granules may be of a uniform size, or of varying sizes.

In some embodiments, the scaffolds include an outer cortex or coating. Formation of an outer cortex or coating on a core component can be performed using any method known to those of ordinary skill in the art. In some embodiments, forming a coating involves dipping or immersing a scaffold in a composition or a plasma spray deposition process.

Therapeutic agents may be added to the scaffolds or incorporated into the scaffolds using any method known to those of ordinary skill in the art. Therapeutic agents include biomolecules. Biomolecules include, e.g., proteins, amino acids, peptides, polynucleotides, nucleotides, carbohydrates, sugars, lipids, glycoproteins, nucleoproteins, lipoproteins, steroids that are commonly found in cells or tissues, whether the molecules themselves are naturally-occurring or artificially created (e.g., by synthetic or recombinant methods). Biomolecules also include, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

Thus, the therapeutic agent may be any agent known to those of ordinary skill in the art. One or more therapeutic agents may be coated on the surface of the scaffold, incorporated into the matrix, incorporated into micro-spheres that are suspended and distributed in the matrix, or the scaffold can be immersed in a composition.

Examples of classes of therapeutic agents include osteogenic, osteoinductive, and osteoconductive agents, anti-cancer substances, antibiotics, anti-inflammatory agents, immunosuppressants, anti-viral agents (including anti-HIV agents), enzyme inhibitors, neurotoxins, opioids, hypnotics, antihistamines, lubricants, tranquilizers, anti-convulsants, muscle relaxants, anti-Parkinson agents, antispasmodics, antibiotics, antiviral agents, antifungal agents, modulators of cell-extracellular matrix interactions including cell growth inhibitors and anti-adhesion molecules, vasodilating agents, inhibitors of DNA, RNA, or protein synthesis, antiypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, prostaglandins, targeting agents, chemotactic factors, receptors, neurotransmitters, proteins, cell response modifiers, cells, peptides, polynucleotides, viruses, vaccines, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, hydroxyapatite, and penetration enhancer, anti-inflammatory agents, growth factors, angiogenic factors, antibiotics, analgesics, chemotactic factors, bone morphogenic protein, and cytokines. Therapeutic agents also include antibiotics, anti-inflammatory drugs, and analgesics.

Non-limiting examples of therapeutic agents include non-collagenous proteins such as osteopontin, osteonectin, bone sialo proteins, fibronectin, laminin, fibrinogen, vitronectin, trombospondin, proteoglycans, decorin, proteoglycans, beta-glycan, biglycan, aggrecan, veriscan, tanascin, matrix gla protein hyaluran, cells; amino acids; peptides; inorganic elements; inorganic compounds; organometallic compounds; cofactors for protein synthesis; cofactors for enzymes; vitamins; hormones; soluble and insoluble components of the immune system; soluble and insoluble receptors including truncated forms; soluble, insoluble, and cell surface bound ligands including truncated forms; chemokines, interleukines; antigens; bioactive compounds that are endocytozed; tissue or tissue fragments; endocrine tissue; enzymes such as collagenase, peptidases, oxidases, etc; polymeric cell scaffolds with parenchymal cells; angiogenic drugs, polymeric carriers containing bioactive agents; encapsulated bioactive agents; bioactive agents in time-release form; collagen lattices, antigenic agents; cytoskeletal agents; cartilage fragments; living cells such as chondrocytes, osteoblasts, osteoclasts, fibroclasts, bone marrow cells, mesenchymal stem cells, etc; tissue transplants; bioadhesives; bone morphogenic proteins (BMPs), transforming growth factors (TGF-.beta.), insulin-like growth factor, platelet derived growth factor (PDGF); fibroblast growth factors (FGF), vascular endothelial growth factors (VEGF), epidermal growth factor (EGF), growth factor binding proteins, e.g., insulin-like growth factors; angiogenic agents; bone promoters; cytokines; interleukins; genetic material; genes encoding bone promoting action; cells containing genes encoding bone promoting action; cells genetically altered by the hand of man; externally expanded autograft or xenograft cells; growth hormones such as somatotropin; bone digestors; anti-tumor agents; fibronectin; cellular attractants and attachment agents; immunosuppressants; bone resorption inhibitors and stimulators; mitogenic factors; bioactive factors that inhibit and stimulate second messenger molecules; cell adhesion molecules, e.g., cell-matrix and cell-cell adhesion molecules; secondary messengers; monoclonal antibodies specific to cell surface determinants on mesenchymal stem cells; portions of monoclonal antibodies specific to cell surface determinants on mesenchymal stem cells; portions of monoclonal antibodies specific to cell surface determinants on mesenchymal stem cells; clotting factors; polynucleotides; and combinations thereof

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed:

1. A bioreactor comprising:
a culture medium;
a scaffold immersed in the culture medium, the scaffold having a plurality of macro-pores, and a plurality of nano-pores; the scaffold having thereon a plurality of cancerous cells selected from the group consisting essentially of:
osteosarcoma cells,
chondrosarcoma cells,
Ewing's sarcoma cells,
fibrosarcoma cells; and
carcinoma cells.

2. The bioreactor of claim 1, wherein substantially all of the macro-pores have a diameter between about 150 μm and about 650 μm.

3. The bioreactor of claim 1, wherein substantially all of the macro-pores have a diameter between about 200 μm and about 400 μm.

4. The bioreactor of claim 1, wherein substantially all of the nano-pores have a diameter between about 100 nm and about 400 nm.

5. The bioreactor of claim 1, wherein the plurality of macro-pores are interconnected.

6. The bioreactor of claim 1, wherein the scaffold further comprises a plurality of micro-channels.

7. The bioreactor of claim 6, wherein substantially all of the micro-channels have a diameter between about 25 μm and 70 μm.

8. The bioreactor of claim 6, wherein the plurality of micro-channels are interconnected.

9. The bioreactor of claim 1, further comprising a perfusion pump operable to circulate the culture medium.

10. The bioreactor of claim 1, the scaffold having thereon a plurality of a combination of healthy cells and cancerous cells.

11. The bioreactor of claim 10, the healthy cells being selected from the group consisting of:
osteoblasts;
osteoblast precursors;
fibroblasts;
muscle cells;
bone marrow cells; and
mesenchymal stem cells.

12. (canceled)

13. The bioreactor of claim 1, the culture medium comprising a pharmaceutical.

14. The bioreactor of claim 13, the pharmaceutical being a chemotherapeutic agent.

15. The bioreactor of claim 1, the scaffold being substantially cylindrical.

16. The bioreactor of claim 1, the scaffold being substantially spherical.

17. The bioreactor of claim 1, the scaffold having a diameter of about 8 mm.

18. The bioreactor of claim 1, the scaffold having a diameter of about 100 μm.

19. The bioreactor of claim 15, the scaffold having a height of about 8 mm.

20. The bioreactor of claim 10, the cells being distributed substantially evenly throughout the scaffold.

21. The bioreactor of claim 20, the scaffold having an interior region, the interior region having hypoxic cells.

22. The bioreactor of claim 1, the scaffold being substantially cuboidal.
